(19) United States
(12) Patent Application Publication
(10) Pub. No.: US 2023/0190586 A1
(43) Pub. Date: Jun. 22, 2023

(54) DENTAL FORMULATION FOR THE TREATMENT OF TOOTH SENSITIVITY

(71) Applicants: M B LLOYD LIMITED, Hebden Bridge (GB); TOM RILEY LIMITED, Hebden Bridge (GB)

(72) Inventors: Matthew Lloyd, Harrogate (GB); Thomas Riley, Yarm (GB); Donncha Haverty, Nenagh (IE)

(21) Appl. No.: 16/093,863

(22) PCT Filed: Apr. 12, 2017
(86) PCT No.: PCT/GB2017/051035

§ 371 Date: Oct. 15, 2018

(30) Foreign Application Priority Data
Apr. 14, 2016 (GB) 1606551.8

(51) Int. Cl. A61K 6/838; A61K 6/17

(52) U.S. Cl. CPC A61K 6/838; A61K 6/17

(57) ABSTRACT

The present invention provides a dental formulation for the treatment of tooth sensitivity, wherein the formulation comprises specifically engineered apatite particles, having an ideal morphology, size and density for the occlusion of dentinal tubules.

### CROSS REFERENCE TO RELATED APPLICATIONS

This application is a National Stage application of PCT/GB2017/051035, filed Apr. 12, 2017, which claims the benefit of Great Britain Application No. 1606551.8, filed Apr. 14, 2016, both of which are incorporated by reference in their entirety herein.

### BACKGROUND

Dentine sensitivity (DS), tooth sensitivity (TS) and or dentinal hypersensitivity (DHS) are used interchangeably in the literature to describe the same clinical oral health problem, namely the pain arising from exposed dentine in response to environmental stimuli, which may be thermal, evaporative, tactile, osmotic or chemical in nature (Bekes, 2015; Gillam and Talioti, 2015). The pain is clinically described as sharp and transient and may be localized or generalized, to one or several teeth respectively. The prevalence of DS has been reported with some considerable variability in the literature but incidences as high as 34% have been diagnosed and the condition is generally accepted by dental professionals to be increasing (Fischer et al., 1992; West et al., 2013). DS is associated with tooth demineralization and the loss of either enamel or cementum to expose underlying dentine. This mineral loss exposes an increased number of dentinal tubules, open microscopic fluid filled cylindrical channels that traverse the dentine from the pulp to either the dentinoenamel junction (DEJ) or the dentinocemental junction (DCJ) in the case of the crown or root of the tooth respectively. These tubules are some 1-5 microns in diameter depending on the severity of the condition. The hydrodynamic theory is the widely accepted mechanism of DS, wherein the fluid movement within the tubules in response to the environmental stimuli causes shear forces to be exerted on mechanoreceptor nerves in the central end of the tubules (Holland, 1994; Li et al., 2013; Mantzourani and Sharma, 2013; Yoshiyama et al., 1996). Studies have revealed that the rate of pulpal nerve stimulation is proportional to the rate of fluid flow within the tubules with those stimuli causing a net outward movement of fluid flow (cold, evaporative and osmotic) generating the most severe pain response, while those connected with net inward fluid flow (heat) are less severe.

The causes of DS are ultimately those that result in demineralisation giving rise to increased exposure of tubules and permeability of the dentine (Bekes, 2015; Hegde et al., 2014; Salas et al., 2015; West and Joiner, 2014; Wongkhantee et al., 2006). The most common causes of demineralisation of the cementum, enamel, or dentine are attrition, abrasion, gingivial recession, and erosion. Attrition is generally attributed to wear resulting from tooth on tooth mechanical action and is distinguished from abrasion which is tooth wear arising from extrinsic causes such as the abrasive action of particles within dentrifice compositions or toothbrushes themselves. In particular whitening toothpastes containing abrasive particles for plaque and stain removal are associated with higher Relative Dentine Abrasivity (RDA) and Relative Enamel Abrasivity (REA) indices. Gingivial recession (CR) is associated with the recession of the soft tissues (gum line) surrounding the teeth which can also lead to the increased exposure of tubules in the roots of the teeth. There are many pathologies that give rise to GR but it has been associated with the abrasive action of Tooth brushes[Sehmi & Olley 2015].

Mineral loss due to erosion is generally ascribed to the action of acid metabolites derived from the microbial breakdown of sugars and residual food particles trapped in or on the teeth. The lowering of the pH locally at the tooth surface causing a dissolution of Ca ions from the natural apatitic structure comprising the enamel and the hard tissue component of the dentine resulting in mineral softening and ultimately loss. However erosion can also be as a consequence of applied chemical treatments such as the use of oxidisers for stain and plaque removal in both clinical and domestic settings. Indeed increased incidences of DS have been associated with the increased use of bleaching agents, such as peroxides and dioxides in cosmetic dentistry (Bonafé et al., 2013; Carey, 2014; Olley et al., 2015).

Allied to the presence of exposed tubules, dentrifice use itself may exacerbate DS. Dentrifices with low pH will accelerate Ca loss and increase exposed tubule diameters, while high molality dentrifices (those containing higher concentrations of soluble salts and consequentially higher osmotic pressures than saliva) will facilitate net outward fluid flow within the tubules generating the characteristic DS pain response.

Other factors that affect tubule exposure and porosity include the dental pellicle which is a layer of proteinaceous material some microns in dimension that is precipitated on the teeth from the salivary fluids within a short time frame of tooth washing, The extent to which the pellicile is periodically removed and replaced is also dependent of the nature of the dentrifice, a characteristic arbitrarily indicated by the (pellicile cleaning rate) PCR. The PCR is dependent on dentrifice composition and allows a comparison of different dentrifices at a given tooth brush configuration and brush stroke rate.

In addition it has been suggested that similar to the smear layer manifest during dental surgery as a consequence of the abrasive mechanical tools used to prepare damaged teeth for fillings etc., that an analogous smear layer comprising abraded hard tissue (bits of enamel, dentine, plaque, etc.) and other material within the dentrifice is formed by the action of certain toothpastes, particularly those containing abrasives.

Irrespective of the multiple interrelated factors that give rise to DS, treatment of the problem usually falls into one of two general strategies, designed to either: 1) stabilize (anaesthetise) the intradental nerves, usually with potassium ions or 2) occlude (block) the dentinal tubules.

Potassium salts have been used as nerve numbing agents in dentifrice since the seventies, when Hoodoo reported the use of 1-15% potassium nitrate solutions for DS reduction. Several other salts of potassium are also effective including potassium chloride, potassium citrate, potassium oxalate, potassium fluoride and potassium bicarbonate indicating that it is the soluble K+ ion that acts upon the pupal nerve. Many dentifrice compositions containing soluble potassium slats have been disclosed in the prior art such as for example U.S. Pat. No. 5,240,697 to Norfteet et al. It has been demonstrated that when applied in a dentritice as potassium nitrate the availability of K at the teeth is short lived and that K is not present in the saliva above its normal levels 20 minutes after application of a 10% dentrifice. Diffusion models predicting K concentration gradients within the tubule also confirm the transient nature of potassium availability when applied as a soluble salt (Stead et al., 1996).

In some sense related many dentrifices also incorporate fluoride in soluble form which exchanges for carbonate or hydroxyl in the native enamel forming fluorapatite at the tooth surface. This provides the benefit of improving the strength of existing enamel while Fluoride ions may also combine with Ca and phosphate in the saliva to precipitate new material. Stannous Fluoride in particular is thought to provide a desensitizing benefit through occlusion but other salts of fluoride have also been used in dentrifices including fluorophosphates and fluorosillicates.

More recently the trend in treatments for DS has been towards the addition of insoluble components in dentrifice compositions, usually nano-dimensioned to effect occlusion of the dentinal tubules. The most commonly used occluding agents are those that are not apatitic themselves, but induce the precipitation of a Calcium phosphate amorphous layer with a Ca/P ratio between 1.3 and 1.7 depending on the biomaterial used. By far the most well-known occluding agent is bioglass 45S5 nanomaterials, the basis of the Novamin technology. The induction of Calcium Phosphate precipitation by bioglass is surface mediated and facilitated by the high solubility of bioglass due to its low silica content (45%) and its relatively high NaO₂, CaO (25%) and Pyrophosphate (6%) content. When bioglass nanoparticles occupy the tubule openings the locally high concentration of Ca and PO4 present at its surface induce the precipitation of the amorphous Calcium phosphate, partially occluding the tubules and providing a desensitizing benefit. The precipitated layer is small even relative to the bioglass particle and kinetically slow to form.

Combinations of lower calcium phosphate salts such as calcium pyrophosphate, mono, di or tri calcium phosphates, octacalcium phosphate (OCP), tetracalcium phosphate (TTCP) and amorphous calcium phosphates (ACP) can effect similar amorphous precipitates through cement reactions as encountered in bone cement chemistry with varying Ca/P ratios manifest, depending on the chemistry of the physiological environment and the solubility product of the combination of Calcium Phosphates used. However combinations of different Calcium phosphates are rarely used in dentrifices (to prevent cement reactions in the tube) and lower calcium phosphate salts when used in dentrifices are usually used individually to provide an abrasive, cleaning benefit.

Properly apatitic materials have been used for occlusion in particular hydroxyapatite, fluoroapatite and Hydroxy-carbonate apatite. The solubility products of calcium apatites are the lowest of all the calcium phosphate salts, crystalline calcium apatite once formed generally being the most thermodynamically stable Calcium phosphate phase at physiological temperature and pH. The biocompatibility of Hydroxyapatite as a biomaterial for orthopaedic and dental applications derives from its similarity to the mineral component of bone or dentine which comprise 60-70% Carbonate substituted Hydroxyapatite. Enamel is 95-98% hydroxyapatite with a very low level of carbonate substitution. Hydroxyapatite is a crystalline solid with the formula Ca₁₀ (PO₄)₆ OH₂ and a characteristic XRD pattern.

When used as occluding agents, apatites and in particular hydroxyapatite is also used as nanocrystallites, consistent with the occlusion mechanism being connected with induced amorphous amorphous phase precipitation on the high surface area nanoparticles. Most hydroxyapatite used in dentrifices is synthesized by wet chemical processes which is a cost effective method of converting suitably chosen soluble salts of calcium and phosphorous mixed in appropriate molar ratios to Hydroxyapatite by precipitation or sol reactions at ordinary temperatures and usually alkaline pH. This generally results in nanocrystallites of angular morphology with plate or columnar like shapes.

It has been shown, that spherical (equiaxed) particles provide better infiltration of dentinal tubules when compared with elongated or angular crystallites (Earl et al., 2009). Consequently some authors have claimed specific advantage by using apatite nanoparticles with columnar morphologies as these have lower aspect ratios than their platelet counterparts (Noerenberg et al., 2008) for example.

To crystallize Hydroxyapatite particles that are greater than nano-dimensioned requires thermal processing, either of appropriate solid precursors ground and mixed prior to heating or by pressing and sintering nanomaterials precipitated by wet chemical processes at temperatures above 800 C (sintering) where grain growth and densification become feasible. This inevitably leads to some degradation of the Hydroxyapatite to lower calcium salts such as TCP and CaO due to hydroxyl loss. In addition, the loss in stoichiometry is usually accompanied by a loss of activity at the surface of these materials as this is where the less bioactive phases are formed during prolonged exposure to high temperature. The materials (sintered bodies) that result can be subsequently milled and classified to hard yield micron particles with good mechanical properties albeit that they will usually be of angular morphology. Such larger micron (>20 micron) Hydroxyapatite is generally utilized in dentrifice for its abrasive cleaning action.

The relationship between particle shape and abrasivity can be quantified by several shape or form factors including the roundness factor (RN), the irregularity Parameter (IP) and the spike parameter (SPQ) to name a few. These parameters measure the departure of 2d projections of the surface of real particles from the ideal surface of a sphere. With perfectly spherical particles having RN, and IP of 1.0 and SPQ of 0.0 (VALDEK and Kaerdi, Helmo, 2001). With increasing departure from the ideal spheroidal shape, these shape and form factors increase. The more removed from the ideal sphere, the more abrasive a particle of given size and material will be.

TABLE 1
Form factors of various shapes

| Shape | | | | | |
|---|---|---|---|---|---|
| Factor | [symbol] | [symbol] | [symbol] | [symbol] | [symbol] |
| RN | 1.0 | 1.055 | 1.103 | 1.273 | 1.654 |
| IP | 1.0 | 1.082 | 1.155 | 1.414 | 2.000 |
| SPQ | 0.0 | 0.383 | 0.500 | 0.707 | 0.866 |

Acceptable RDA values for dentrifices limit the concentrations and nature of materials that can be used as abrasives and cleaning agents. Processes are available to reduce the abrasivity of ceramic micron particles derived from the thermal sintering and milling processes described above involving partially melting the outside surface of the aerosolised irregularly shaped particles in a flame. Such processes are claimed to reduce the abrasivity of Alumina and silica beneficially for use in dentrifices (Deckner et al., 2014; Lucas, 2015). However, most likely as a consequence of the processing costs the use of microcrystalline sphericised Hydroxyapatite synthesized in this way is not prevalent in dentrifices, and where dense micron spheres are used as abrasives, alumina and silica are the most common material choices.

Low density (high porosity) microspheres of HA can be made by agglomerating nano-crystallites from standard wet chemical processes into micron sized secondary spheres comprising the primary nanocrystallites. Processes to achieve this include spray drying of nano-slurries typically at temperatures up to 250 C or by the co-solvent micro-emulsion techniques. The temperature regimes during such operations result in low density (high surface area) secondary microparticles with limited contact between the primary nanoparticles comprising the aggregates, which form a nanostructured porous network that is mechanically weak. Dentrifice compositions comprising such hydroxyapatite particles are disclosed in the prior art, for example (Hill et al., 2014).

Secondary microspheres derived from nano-slurries can of course be subsequently sintered to improve mechanical strength but in the absence of pressing (standard sintering processes) to maximize contact between individual crystallites little grain growth and densification occurs; and the particles remain porous and mechanically fragile. Thus it is not trivial to produce Hydroxyapatite particles that are both spherical and strong, which is to say Hydroxyapatite particles that are spherical, micron dimensioned, dense and microcrystalline as opposed to nanocrystalline. Density in the context of the individual particles means an individual particle density that is higher than 90% of the theoretical density of single crystal Hydroxyapatite. Powders with micron particles of this nature have a bulk powder density of 1.0 Kg/Lt or more. By comparison the hulk powder density of nano or nano-structured particles is considerably less typically 0.2 Kg/Lt to 0.4 Kg/Lt. It is particularly difficult to make Hydroxyapatite particles that are of 0.8-3 micron dimension (diameter of dentinal tubules) that have the properties alluded to above. Consequently almost all hydroxyapatite used in dentrifices for occlusion purposes is nano-hydroxyapatite whether fully dispersed or in the form of agglomerates with secondary structure.

Particle size and shape alone is not the only consideration in the engineering of an optimum occluding agent, when spherical silica nanoparticles are used as possible occluding agents to block dentinal tubules aggregation of the particles is required to secure any substantial occlusion (coverage) of the tubule openings which are some two orders of magnitude larger than the dimensions of the nanoparticles typically used for that purpose. It has been demonstrated in occlusion tests using nanobioglass on pre-etched (no smear or pellicle layer over the tubule opening) tooth surfaces in a laboratory setting, that to effect sufficient aggregation of the particles to cover the tubule openings to any significant extent, the chemistry of the individual particle surfaces had to be modified with surfactants (Claire et al., 2015). Even with the use of appropriate modifications to the surface chemistry the aggregates formed are mechanically weak and in an in vivo setting would be unlikely to be mechanically strong enough to penetrate smear or pellicle layers and be pushed into dentinal tubules under real life conditions.

While the provision of Hydroxyapatite particles with appropriately engineered morphology, density and dimension would be of obvious benefit in improving the occlusion properties of Hydroxyapatite used in desensitizing dentrifices the inclusion of K for simultaneous nerve stabilization is also desirable.

Several dentrifices are disclosed that claim a benefit from compositions containing both apatitic materials in combination with a potassium compound (Deckner et al., 2014; Hill et al., 2014; Saito, T, 2015.). However in all cases the particle properties of the apatite are not specifically engineered for optimum occlusion and the potassium is a separate ingredient that takes the form of a soluble potassium salt. While the apatite is an insoluble component and contributes little to the osmotic pressure of the dentrifice the soluble potassium salt will contribute significantly to the molality of the dentrifice generating a net outward movement of the fluid within the tubules. In addition, and also directly related to solubility, the availability of K for nerve stabilization is transient.

More robust mechanisms to deliver potassium ions are thus required that would provide K directly within the tubule in a more sustained concentration without adversely affecting the molality of the dentrifice.

One possible strategy would be to deliver the potassium as a component in an apatite. Indeed the apatites are known for their capacity to undergo a number of cationic substitutions to yield a range of substituted apatites with the general formula Ca₅₋ₓ Mₓ (PO₄)₃OH, where M is a divalent cation. Other members of the alkaline earth metals Mg, Ba, Sr are readily substituted for Ca to a high degree. Other transition divalent metals such as Zn, Ag, and Sn, can also be readily substituted up to an upper limit of about x=0.3 without significant disruption to the apatitic structure.

Incorporation of monovalent ions (K, Na etc.) into the apatitic lattice is however not as straightforward. Previous attempts to incorporate potassium ions in Hydroxyapatite through conventional synthesis routes (wet chemical, solid state synthesis) generally result in the potassium being incorporated in lower calcium phosphate phases that form as a consequence of the attempted substitution, and as a result of the stabilization of less thermodynamically favourable Calcium Phosphate phases formed en route to Hydroxyapatite by conventional wet chemical and sintering synthesis techniques. Most usually Brushite or its dehydrated equivalent (post sintering) calcium pyrophosphate is the phase that is formed and contains the K. Several successful attempts to synthesize potassium substituted Brushite/Calcium Pyrophosphate have been reported in the literature and analogously attempts to incorporate potassium in Hydroxyapatite have always resulted in the introduction of an additional Brushite phase containing the potassium. Brushite is used in orthopaedics as a bone cement component as it will undergo several hydration and hydrolysis reactions to form hard setting materials such as Tricalcium Phosphate (TCP), Dicalcium Phosphate (OCP) and CaO. Consequently brushite is not he a suitable ingredient or indeed potassium carrier for dentrifices which are ordinarily water based suspensions or emulsions.

If K could be incorporated into the Hydroxyapatite lattice as a solute ion without the formation of the Brushite phase however this potassium would be available to slowly leach out of apatite. If the apatite was further available as stable particles in microsphere form of the correct dimensions, and sufficiently strong to avoid the stresses encountered during the rigours of brushing that they could penetrate any smear or pellicle layers present at the tooth surface, then such apatite particles would provide significant desensitizing benefit being the ideal agent to:

1) provide complete occlusion of exposed dentinal tubules

2) concomitantly provide a source of K within the tubule to stabilize nerves on a prolonged basis

3) provide cleaning benefit without adversely affecting the RDA of the dentrifice

Haverty et al. developed a novel flame spray synthesis (FSS) technology that allows the manufacture of spherical Hydroxyapatite that is also dense and microcrystalline (Haverty, 2012). Additionally this manufacturing method allows the manufacture of many Apatitic solid solutions without the introduction of additional calcium phosphate phases. This methodology involves mixing a Calcium precursor solution and a Phosphorous precursor solution which typically comprise a calcium salt and a phosphorous source dissolved in appropriate solvents respectively. These precursors are mixed prior to the injection of the mixture into a flame in a ratio that provides the requisite Ca/P molar ratio of 1.667 in the flame. The generation of Hydroxyapatite in the flame is facilitated by the presence of catalytic amounts of Hydrogen peroxide added to either precursor. To manufacture potassium doped HA the Calcium precursor solution is augmented with a soluble potassium salt such that the molar ratio of K to Ca in the precursor is in the range 0.001% to 20%.

### SUMMARY

The present application is directed toward the provision of a dental formulation containing such specifically engineered apatite particles, having an ideal morphology, size and density for the occlusion of dentinal tubules while simultaneously having incorporated into the apatitic structure solute potassium ions.

Disclosed is a dental formulation for the treatment of tooth sensitivity including up to 50% by weight of one or more solid solutions, the one or more solid solutions including a solvent component and a solute component, wherein the solvent component is selected from hydroxyapatite, fluorapatite, oxyapatite, chlorapatite, substituted apatites, or mixtures thereof; wherein the solute component includes potassium ions, wherein the one or more solid solutions are inclusive of substantially spherical particles, wherein the particles include a single microcrystalline phase; and wherein at least 5% of the particles are below 3 microns in size.

### BRIEF DESCRIPTION OF THE FIGURES

The advantages and embodiments of the present application will be understood more clearly by reference to the accompanying drawings:

FIG. 1. Schematic representation of the potential desensitizing action of different particle types on open tubules at the tooth surface.

FIG. 2. Scanning electron Micrographs (SEM) and Focused Ion Beam (FIB) image detailing of FSS Hydroxyapatite microspheres.

FIG. 3. Scanning electron Micrographs (SEM) particulate Hydroxyapatite comprising aggregated primary nanocrystallites.

FIG. 4A pair of xrd patterns detailing the difference between FSS Hydroxyapatite microparticles and the aggregated nanocrystallites.

FIG. 5A pair of XRD patterns and an SEM micrograph detailing the distinction between Apatitic solid solutions containing potassium manufactured by FSS and typical synthesis techniques.

### DETAILED DESCRIPTION

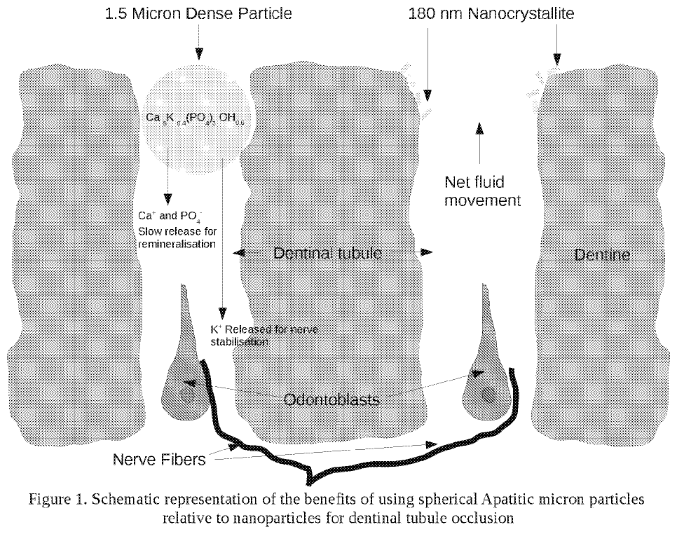

The similarity of Hydroxyapatite and its fluoride, chloride or carbonate substituted analogues to enamel and the mineral content of dentine makes it the material of choice for remineralisation and dentinal tubule occlusion for the treatment of DS. The ideal apatite particles for dentinal tubule occlusion are substantially spherical with a diameter approximately equal to or slightly smaller than the tubule diameters (1-3 micron). Additionally these particles are ideally dense and microcyrstalline providing the further advantage of being mechanically stable enough to withstand the mechanical stresses encountered during application and to penetrate any smear or pellicle layers encountered on the teeth. A further improvement in the desensitizing ability of the ideal apatite particles for desensitizing formulations is provided by the incorporation of K ions in the apatitic lattice as a solute without the formation of other Calcium Phosphate phases. The benefit of using such particles to occlude dentinal tubules relative to nanocrystallites is represented schematically in FIG. 1.

Apatitic particles with these characteristics have to date not been used to provide a desensitizing benefit in dental formulations for the treatment of tooth sensitivity. However by using such specifically engineered apatitic particles a significant benefit in the treatment of tooth sensitivity is achievable relative to the use of nanostructured apatitic materials and or soluble potassium salts.

The present invention provides a dental formulation for the treatment of DS that contains up to 50% by mass of an apatitic solid solution, the solid solution comprising particles with specific morphology, size and crystallinity.

Therefore, in accordance with the present invention, there is provided a dental formulation for the treatment of tooth sensitivity comprising:

up to 50% by weight of one or more solid solutions, the one or more solid solutions comprising a solvent component and a solute component, wherein

the solvent component is selected from hydroxyapatite, fluorapatite, oxyapatite, chlorapatite, substituted apatites, or mixtures thereof;

wherein the solute component comprises potassium ions;

wherein the one or more solid solutions are comprised of substantially spherical particles, wherein the particles comprise a single microcrystalline phase; and

wherein at least 5% of the particles are below 3 microns in size.

The dental formulation is selected from a dentrifice, a gel, a toothpaste, a mouth wash or mouth rinse, or a dental strip.

Typically, the solid solution has a molar ratio of potassium to calcium that is less than or equal to about 0.15.

According to one embodiment of the invention, the calcium ions in the apatite in the formulation may be substituted by one or more different divalent cations chosen from Mg, Ba, Sr, Zn, Ag or Sn.

In preferred embodiments of the invention the apatitic solid solution comprises an apatitic solvent with potassium.

In one embodiment the solid solution is an apatitic material with the general formula

Ca₁₀ Kₓ (PO₄)₆ A₂₋ₓ Bₓ

Where A is OH (hydroxyl), F (fluoride), Cl (Chloride) or I (iodide), B is CO₃ (Carbonate) O (Oxy) and the solute potassium ions occupy the interstitials of the apatitic lattice. In preferred embodiments X is in the range 0.01 to 1.5 and the K/Ca molar ratio is in the range 0.001 to 0.15. The Ca/P molar ratio is close to that of a pure Calcium apatite 1.67. In a preferred embodiment X is in the range 0.5 to 1.0 and the K/Ca molar ratio is 0.05 to 0.1.

In one embodiment the solid solution is an apatitic material with a fraction of its Ca content substituted with other divalent ions, the solid solution has the general formula:

[Ca_{1-w} M_{w}]₁₀ Kₓ (PO₄)₆ A₂₋ₓ Bₓ

Where A is OH (hydroxyl), F (fluoride), CI (Chloride) or I (iodide), B is CO₃ (Carbonate) or O (Oxy) and the solute potassium ions occupy the interstitials of the apatitic lattice. M is a divalent cation chosen from Mg, Ba, Sr, Zn, Ag or Sn and the fraction of calcium substitution w is between 0 and 0.1. In preferred embodiments X is in the range 0.01 to 1.5 and the K/(Ca+M) molar ratio is in the range 0.001 to 0.15. The (Ca+M)/P molar ratio is close to that of a pure Calcium apatite 1.67. In a preferred embodiment X is in the range 0.5 to 1.0 and the K/(Ca+M) molar ratio is 0.05 to 0.1.

In a further embodiment of the present invention the solid solution is an apatitic material with one of the following general formulas:

[Ca_{1-w} M_{w}]₁₀ Kₓ [(PO₄)_{1-Z} (SiO₄)_{Z}]₆ A_{2-X-6Z} B_{X}

[Ca_{1-w} M_{w}]₁₀ Kₓ [(PO₄)_{1-Z} (SiO₄)_{3Z/4}]₆ A_{2-X} B_{X}

[Ca_{1-w} M_{w}]₁₀ Kₓ (PO₄)_{6-X} (SiO₄)_{X} A_{2-Y} B_{Y/2}

Where A is OH (hydroxyl), F (fluoride), Cl (Chloride) or I (iodide), B is CO₃ (Carbonate) or O (Oxy) and the solute potassium ions occupy the interstitials of the apatitic lattice. M is a divalent cation chosen from Mg, Ba, Sr, Zn, Ag or Sn and the fraction of calcium. substitution w is between 0 and 0.1. Z is the fraction of Phosphate substitution by Tetravalent silicate and is in the range 0 to 0.1. In preferred embodiments X is in the range 0.01 to 1.5 and the K/(Ca+M) molar ratio is in the range 0.001 to 0.15. The (Ca+M)/P molar ratio is higher than that of a pure Calcium apatite and is in the range 1.67 to 1.89. In a preferred embodiment X is in the range 0.5 to 1.0 and the K/(Ca+M) molar ratio is 0.05 to 0.1. Y has a maximum possible value of 2.

In a further embodiment of the present invention the solid solution is an apatitic material with the general formula:

[Ca_{1-w} M_{w}]₁₀ Kₓ [(PO₄)_{1-z} D_{z}]₆ A_{2-x-6z} B_{x+6z}

Where A is OH (hydroxyl), F (fluoride), Cl (Chloride) or I (iodide), B is CO₃ (Carbonate) or O (Oxy) and the solute potassium ions occupy the interstitials of the apatitic lattice. D is a divalent anion chosen from CO₃ carbonate, or SO₄ (sulphate) and z is between 0 and 0.2. M is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18. The (Ca+M)/(P) molar ratio is in the range 1.66 to 1.89. In a preferred embodiment X is in the range 0.5 to 1.2 and the K/(Ca+M) molar ratio is 0.05 to 0.14. Y has a maximum possible value of 2.

In a further embodiment of the present invention the solid solution is an apatitic material with the general formula:

[Ca_{1-w} M_{w}]₁₀ Kₓ [(PO₄)_{1-z} (HCO₃)_{z}]₆ A_{2-x-12z} B_{x+12z}

Where A is OH (hydroxyl), F (fluoride), Cl (Chloride) or I (iodide), B is CO₃ (Carbonate) or O (Oxy) and the solute potassium ions occupy the interstitials of the apatitic lattice. Z is the degree of substitution of phosphate by the monovalent anion HCO₃ (hydrogenearbonate), and z is between 0 and 0.2. N is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18. The (Ca+M)/(P) molar ratio is in the range 1.66 to 1.89. In a preferred embodiment X is in the range 0.5 to 1.2 and the KI(Ca+M) molar ratio is 0.05 to 0.14. Y has a maximum possible value of 2.

In a further embodiment of the present invention the solid solution is an apatitic material with the general formulas:

[Ca_{1-w} M_{w}]₁₀₋ₓ Kₓ [(PO₄)_{1-z} (SiO₄)_{z}]₆A_{2-6z-x-y} B_{y/2}

[Ca_{1-w} M_{w}]₁₀₋ₓ Kₓ (PO₄)₆₋ₓ (SiO₄)ₓ A_{2(1-x)-y} B_{y/2}

Where A is OH (hydroxyl), F (fluoride), Cl. (Chloride) or I (iodide), B is CO₃ (Carbonate) or O (Oxy) and the solute potassium ions occupy Ca positions in the apatitic lattice. Z is the degree of substitution of phosphate by the Tetravalent SiO4 (Silicate), and z is between 0 and 0.2. M is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18. The (Ca+M)/(P) molar ratio is in the range 1.66 to 1.89. In a preferred embodiment X is in the range 0.5 to 1.2 and the K/(Ca+M) molar ratio is 0.05 to 0.14. Y has a maximum possible value of 2.

In a further embodiment of the present invention the solid solution is an apatitic material with the general formulas:

[Ca_{1-w} M_{w}]₁₀₋ₓ Kₓ [(PO₄)_{1-z} (D)_{z}]₆ A_{2-6z-x} B_{y/2+6z}

[Ca_{1-w} M_{w}]₁₀₋ₓ Kₓ (PO₄)₆₋ₓ Dₓ A_{2-y} B_{y/2}

Where A is OH (hydroxyl), F (fluoride), Cl (Chloride) or I (iodide), B is CO₃ (Carbonate) or O (Oxy) and the solute potassium ions occupy Ca positions in the apatitic lattice. D is a divalent anion chosen from CO₃ carbonate, or SO₄ (sulphate) and z is between 0 and 0.2. M is a divalent cation chosen from Mg, Ba, Sr, Zn, Ag or Sn and w is between 0 and 0.1. In preferred embodiments X is in the range 0.01 to 1,5 and the K/Ca molar ratio is in the range 0.001 to 0.18. The (Ca+M)/(P) molar ratio is in the range 1.66 to 1.89. In a preferred embodiment X is in the range 0.5 to 1.2 and the K/(Ca+M) molar ratio is 0.05 to 0.14. Y has a maximum possible value of 2.

In a further embodiment of the present invention the solid solution is an apatitic material with the general formulas:

[Ca_{1-w} M_{w}]₁₀₋ₓ Kₓ [(PO₄)_{1-z} (HCO₃)_{z}]₆ A_{2-12z-x-y} B_{y/2+12z}

[Ca_{1-w} M_{w}]₁₀₋ₓ Kₓ (PO₄)_{6-x/2} (HCO₃)_{x/2} A_{3-y} B_{y/2}

Where A is OH (hydroxyl), F (fluoride), Cl (Chloride) or I (iodide), B is CO₃ (Carbonate) or O (Oxy) and the solute potassium ions occupy Ca positions in the apatitic lattice. Z is the degree of substitution of phosphate by the monovalent anion HCO₃ (hydrogencarbonate), and z is between 0 and 0.2. M is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18. The (Ca+M)/(P) molar ratio is in the range 1.66 to 1.89. In a preferred embodiment Xis in the range 0.5 to 1.2 and the K/(Ca+M) molar ratio is 0.05 to 0.14, Y has a maximum possible value of 2.

In a further embodiment of the present invention the solid solution is an apatitic material with the general formulas:

[Ca_{1-w} M_{w}]₁₀ Kₓ [(PO₄)_{1-z-v} D_{z} (HCO₃)ᵥ]₆₋ₓ (SiO₄)ₓ A_{2-6z-12v-y} B_{x+6z+12v+y/2}

[Ca_{1-w}M_{w}]₁₀ Kₓ [(PO₄)_{1-z-u-v} (SiO₄)ᵤ D_{z} (HCO₃)ᵥ]₆ A_{2-x-6z-12v-6u-y} B_{x+6z+12v+y/2}

Where A is OH (hydroxyl), F (fluoride), Cl (Chloride) or I (iodide), B is CO₃ (Carbonate) or O (Oxy) and the solute potassium ions occupy the interstitials of the apatitic lattice. D is a divalent anion chosen from CO₃ carbonate, or SO₄ (sulphate) and z is between 0 and 0.2. U is the degree of substitution of phosphate by the Tetravalent SiO4 (Silicate), and u is between 0 and 0.2. V is the degree of substitution of phosphate by the monovalent anion HCO₃ (hydrogencarbonate), and v is between 0 and 0.2. M is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18. The (Ca+M)/(P) molar ratio is in the range 1.66 to 1.89. In a preferred embodiment X is in the range 0.5 to 1.2 and the K/(Ca+M) molar ratio is 0.05 to 0.14. Y has a maximum possible value of 2.

In a further embodiment of the present invention the solid solution is an apatitic material with the general formula:

[Ca_{1-w} M_{w}]₁₀₋ₓ Kₓ [(PO₄)_{1-z-u-v} (SiO₄)ᵤ D_{z} (HCO₃)ᵥ]₆ A_{2-x-6z-12v-6u-y} B_{6z+12v+y/2}

[Ca_{1-w} M_{w}]₁₀₋ₓ Kₓ [(PO₄)₁₋ᵤ₋ᵥ (SiO₄)ᵤ (HCO₃)ᵥ]₆₋ₓ Dₓ A_{2-2v(6-x)-u(6-x)-y} B_{2v(6-x)+y/2}

[Ca_{1-w} M_{w}]₁₀₋ₓ Kₓ [(PO₄)_{1-u-z} (SiO₄)ᵤ D_{z}]_{6-x/2} (HCO₃)_{x/2} A_{2-6(z+u)+x(z+u)/2} B_{(12z-x)/2+y/2}

Where A is OH (hydroxyl), F (fluoride), Cl (Chloride) or I (iodide), B is CO₃ (Carbonate) or O (Oxy) and the solute potassium ions occupy Ca positions in the apatitic lattice. D is a divalent anion chosen from CO₃ carbonate, or SO₄ (sulphate) and z is between 0 and 0.2. U is the degree of substitution of phosphate by the Tetravalent SiO4 (Silicate), and u is between 0 and 0.2. V is the degree of substitution of phosphate by the monovalent anion HCO₃ (hydrogencarbonate), and v is between 0 and 0.2. M is a divalent cation chosen from Mg, Ba, Sr, Zn, Ag or Sn and w is between 0 and 0.1. In preferred embodiments X is in the range 0.01 to 1.5 and the K/Ca molar ratio is in the range 0.001 to 0.18, The (Ca+M)/(P) molar ratio is in the range 1.66 to 1.89. In a preferred embodiment X is in the range 0.5 to 1.2 and the K/(Ca+M) molar ratio is 0.05 to 0.14. Y has a maximum possible value of 2.

In any of the formulations according to the invention, the one or more phosphate anions may be replaced by one or more different anions in the apatite lattice; the one or more different anions may be selected from, for example, carbonate, hydrogen carbonate or silicate anions.

In the embodiments of the present invention the solid solution is provided in powder form and the morphology of the particles are substantially spherical defined in that the average Roundness (RN) and Irregularity Parameter (IP) shape factors of the particles are both less than 1.10. In preferred embodiments the Roundness (RN) and Irregularity Parameter (IP) shape factors of the particles are both less than 1.05.

In preferred embodiments at least 60% of the particles comprising the apatitic solid solution are dense with no nanoporosity, defined in that the powder is microcrystalline as indicated by X-ray diffraction (XRD). More preferably at least 75% of the particles comprising the apatitic solid solution are dense with no nanoporosity and most preferably at least 90% of the particles comprising the apatitic solid solution are dense with no nanoporosity.

In preferred embodiments of the invention the powder comprising the apatitic solid solution has a D50 (median particle diameter) of not more than 10 micron and at least 5% of the powder particles are below 3 micron diameter. More preferably the powder comprising the apatitic solid solution has a D50 of not more than 7.5 micron and at least 10% of the particles are below 3 micron diameter. Most preferably the powder comprising the apatitic solid solution has a D50 of not more than 5 micron and at least 15% of the powder particles are below 3 micron diameter. The particle diameter refers herein to the distance across a particle at its widest point.

In preferred embodiments of the present invention the dental formulation contains between 0.1% and 50% by mass of the apatitic solid solution. More preferably the dental formulation contains between 10% and 40% by mass of the apatitic solid solution, and most preferably the dental formulation contains between 20% and 30% by mass of the apatitic solid solution.

The apatitic solid solutions of the present invention can be used to provide a desensitizing benefit in several different types of dental formulations. Preferred dental formulations include dentrifices, toothpastes creams and gels.

The dental formulation of the present invention will contain other conventional ingredients well known to those skilled in art depending on the form of the dental product. For instance, in the case of an oral product in the form of a dentifrice cream or paste, the product will comprise an humectant-containing liquid phase and optionally binders and or thickeners which act to maintain the particulate in suspension. A surfactant and a flavouring agent are also usual ingredients of commercially acceptable dentifrices.

Humectants commonly used are glycerol and sorbitol syrup (usually comprising an approximately 70% solution). However, other humectants are known to those in the art, including propylene glycol, lactitol and hydrogenated cornsyrup. The amount of humectant will generally range from about 10 to 85% by weight of the dentifrice. The remainder of the liquid phase will consist substantially of water.

Numerous binding or thickening agents have been indicated for use in dentifrices, preferred ones being sodium carboxymethylcellulose and xanthan gum. Others include natural gum binders such as gum tragacanth, gum karaya and gum arabic, Irish moss, alginates and carrageenans. Silica thickening agents include the silica aerogels and various precipitated silica's. Mixtures of binding and thickening agents may be used. The amount of binder and thickening agent included in a dentifrice is generally between 0.1 and 10% by weight.

It is usual to include a surfactant in a toothpaste and again the literature discloses a wide variety of suitable materials. Surfactants which have found wide use in practice are sodium lauryl sulphate, sodium dodecylbenzene sulphonate and sodium lauroylsarcosinate. Other anionic surfactants may be used as well as other types such as cationic, amphoteric and non-ionic surfactants. Surfactants are usually present in an amount of from 0.5 to 5% by weight of the dentifrice.

Flavours that are usually used in dentifrices are those based on oils of spearmint and peppermint. Examples of other flavouring materials used are menthol, clove, wintergreen, eucalyptus and aniseed. An amount of from 0.1% to 5% by weight is a suitable amount of flavour to incorporate in a dentifrice.

The oral compositions of the invention may also comprise a proportion of a supplementary abrasive agent such as silica, alumina, hydrated alumina or calcium carbonate.

The oral composition of the invention may include a wide variety of optional ingredients, These include an anti-plaque agent such as an antimicrobial compound, for example chlorhexidine or 2,4,4-min-trichloro-2-min-hydroxy-diphenyl ether, or a zinc compound; an anti-tartar ingredient such as a condensed phosphate, e.g. an alkali metal pyrophosphate, hexametaphosphate or polyphosphate or zinc citrate, a fluorine-containing compound such as sodium fluoride or sodium monofluorophosphate; sweetening agent such as saccharin; an opacifying agent, such as titanium dioxide, a preservative, such as formalin; a colouring agent; or pH-controlling agent such as an acid, base or buffer, such as benzoic acid.

According to another embodiment of the invention, the formulation may also include one or more additional components selected from bleaching agents, flavourings agents, stabilisers, viscosity modifiers, antimicrobials or fillers, or a combination thereof.

The bleaching agent may be selected from one or more of arginine carbamide peroxide, hydrogen peroxide, sodium hydroxide, and other peroxide containing products,

The flavouring or sweetening agent may be selected from one or more of mint, cinnamon, vanilla, xylitol, sucralose, sodium saccharin, and menthol.

The stabiliser may be selected from one or more of carrageenan, soybean hemicellulose, dicalcium diphosphate, sodium triphosphate, and citric acid esters.

The viscosity modifier may be selected from one or more of xanthan gum, cellulose gum, seaweed gum, glycerol, glycol and sorbitol.

The antimicrobial may be selected from one or more of zinc citrate, triclosan, glucose oxidase, sodium fluoride, and sodium monofluorophosphate.

The filler may be selected from one or more of calcium carbonate, hydrated silica, and sodium bicarbonate.

Also provided in conjunction with the present invention is a method for the manufacture of a dental formulation according to any preceding a claim, for the treatment of tooth sensitivity comprising combining:

up to 50% by weight of one or more solid solutions, the one or more solid solutions comprising a solvent component and a solute component, wherein the solvent component is selected from hydroxyapatite, fluorapatite, oxyapatite, chlorapatite, substituted apatites, or mixtures thereof;

wherein the solute component comprises potassium ions;

wherein the one or more solid solutions are comprised of substantially spherical particles, wherein the particles comprise a single microcrystalline phase; and

wherein at least 5% of the particles are below 3 microns in size.

The invention will now be illustrated further by the following Examples, which are intended to be illustrative only and in no way limiting upon the scope of the invention.

### EXAMPLE 1

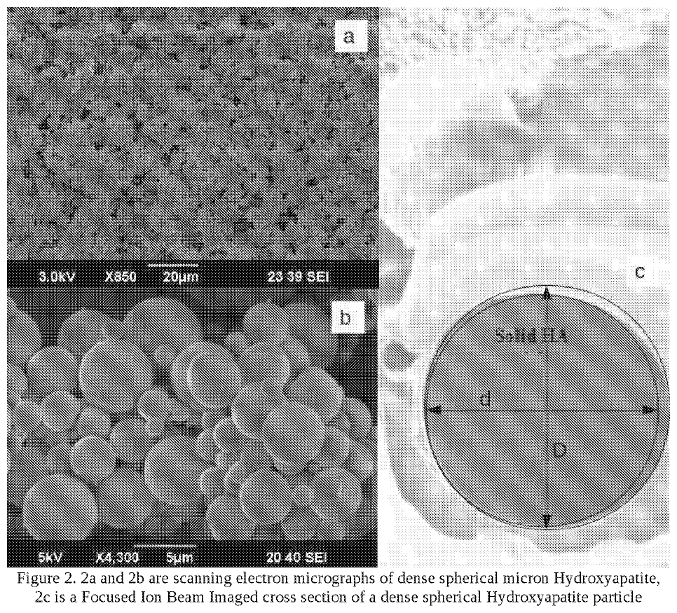

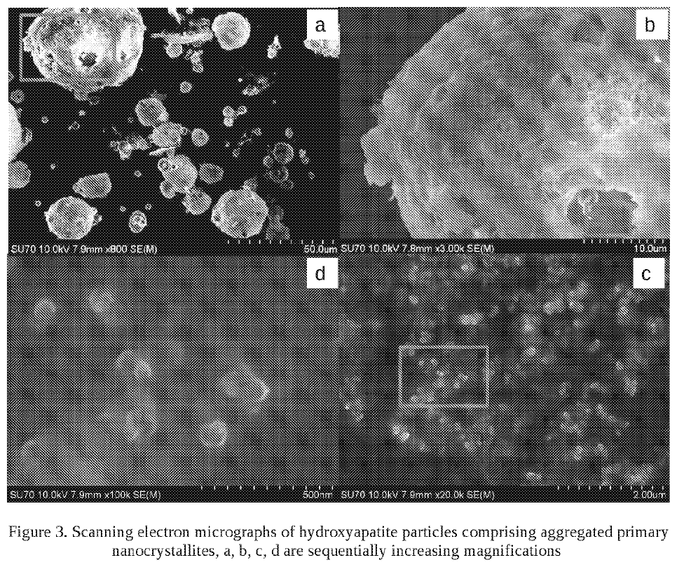

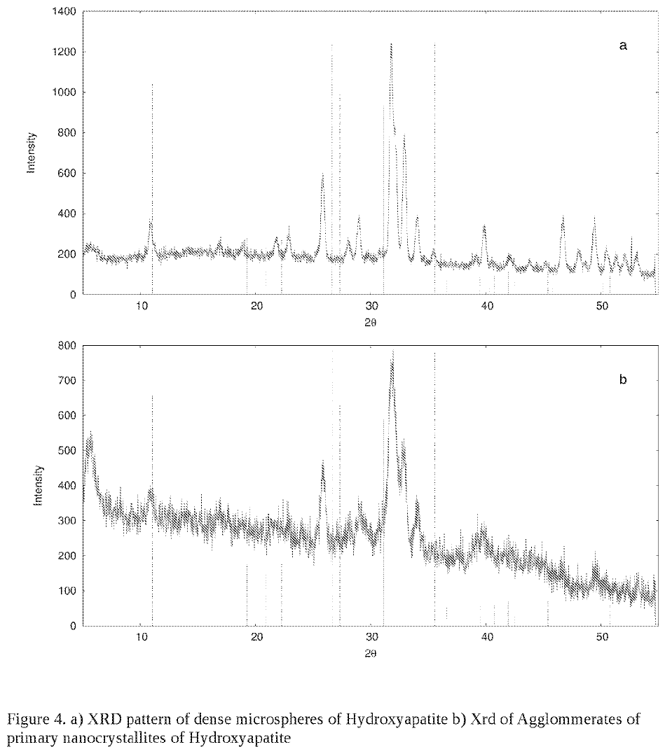

Premier Biomaterials were tasked with synthesizing spherical dense Hydroxyapatite suitable for use in the present invention by their FSS method. This material is compared with a typical Hydroxyapatite particle comprising an aggregation of primary nanocrystallites in FIGS. 2, 3 and 4. Panels 2a and 2b show the morphology of FSS Hydroxyapatite and 2c shows a FIB image respectively of one particle indicating the dense nature of same. Hydroxyapatite particles of this type are considered most suitable for the present application due to their mechanical stability as compared to microspheres comprised of aggregated primary nanocrystallites manufactured by typical wet precipitation and sintering techniques. FIG. 3 shows a series of micrographs of agglomerates comprising primary nanocrystallites with increasing magnification to reveal the primary structure of the micron particle. A distinction between the two types of particulate is clearly manifest in the XRD patterns of both materials wherein the Scherrer line broadening associated with the primary nanocrystallite size (FIG. 4h) is clearly evident in the XRD pattern of the nanostructured particles, while the microcrystalline dense nature of the FSS particles is manifest in the XRD of the FSS particles (FIG. 4a).

### EXAMPLE 2

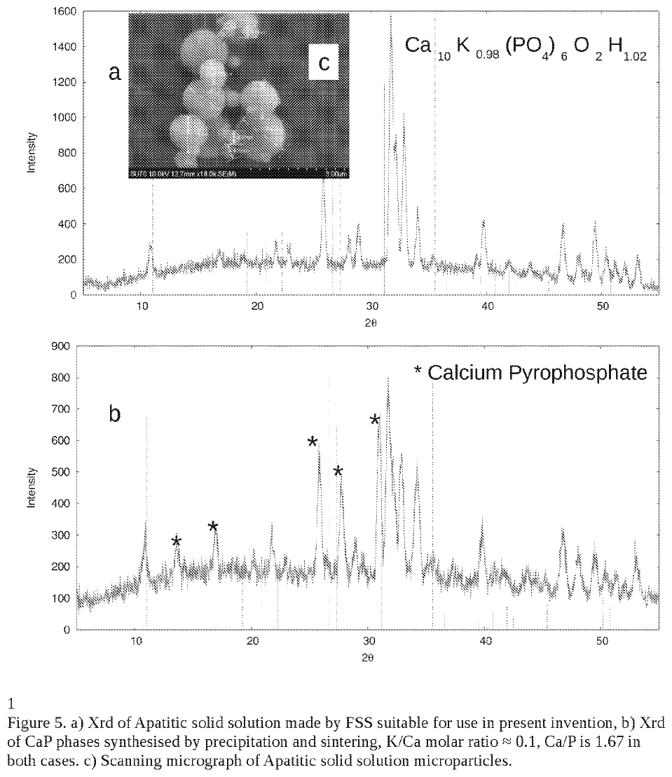

Premier Biomaterials were further tasked with incorporating potassium into the apatitic structure without inducing the precipitation of other calcium phosphate phases. FIG. 5 compares the XRD patterns of K-containing apatite manufactured by FSS and by standard mixing at ordinary temperatures and sintering. The same precursor mix was used in both cases. However the FSS method clearly formed only an apatitic structure without additional phases (5a) while the other process formed additional Ca₂P₂O₇ phases. The insert micrograph (5c) indicates that the spherical structure and dense nature of the particles is maintained with the incorporated potassium.

1. A Dental formulation for the treatment of tooth sensitivity comprising:
up to 50% by weight of one or more solid solutions, the one or more solid solutions comprising a solvent component and a solute component, wherein
the solvent component is selected from hydroxyapatite, fluorapatite, oxyapatite, chlorapatite, substituted apatites, or mixtures thereof;
wherein the solute component comprises potassium ions;
wherein the one or more solid solutions are comprised of substantially spherical particles,
wherein the particles comprise a single microcrystalline phase; and
wherein at least 5% of the particles are below 3 microns in size.

2. The formulation of claim 1 wherein the solid solution has a molar ratio of potassium to calcium that is less than or equal to 0.15.

3. The formulation of claim 1, wherein the D50 value of the particles is not more than 10 microns.

4. The formulation of claim 1, wherein the dental formulation is a dentrifice, a gel or a dental strip.

5. The formulation of claim 1, wherein one or more Calcium ions in the apatite lattice are replaced by one or more different divalent cations.

6. The formulation of claim 5, wherein the divalent cation is selected from Zn, Ag, Sn.

7. The formulation of claim 1 wherein one or more phosphate anions are replaced by one or more different anions in the apatite lattice.

8. The formulation of claim 7, wherein the one or more different anions are selected from carbonate, hydrogen carbonate or silicate anions.

9. The formulation of claim 1, wherein the formulation also includes one or more additional components selected from bleaching agents, flavourings agents, stabilisers, viscosity modifiers, antimicrobials or fillers.

10. The formulation of claim 9 wherein the bleaching agent is chosen from one or more of arginine carbamide peroxide, hydrogen peroxide, sodium hydroxide, and other peroxide containing products.

11. The formulation of claim 9 wherein the flavouring or sweetening agent is chosen from one or more of mint, cinnamon, vanilla, xylitol, sucralose, sodium saccharin, and menthol.

12. The formulation of claim 9 wherein the stabiliser is chosen from one or more of carrageenan, soybean hemicellulose, dicalcium diphosphate, sodium triphosphate, and citric acid esters.

13. The formulation of claim 9 wherein the viscosity modifier is chosen from one or more of xanthan gum, cellulose gum, seaweed gum, glycerol, glycol and sorbitol.

14. The formulation of claim 9 wherein the antimicrobial is chosen from one or more of zinc citrate, triclosan, glucose oxidase, sodium fluoride, and sodium monofluorophosphate.

15. The formulation of claim 9 wherein the filler is chosen from one or more of calcium carbonate, hydrated silica, and sodium bicarbonate.

16. A method for the manufacture of a dental formulation according to claim 1, for the treatment of tooth sensitivity comprising combining:
up to 50% by weight of one or more solid solutions, the one or more solid solutions comprising a solvent component and a solute component, wherein the solvent component is selected from hydroxyapatite, fluorapatite, oxyapatite, chlorapatite, substituted apatites, or mixtures thereof;
wherein the solute component comprises potassium ions;
wherein the one or more solid solutions are comprised of substantially spherical particles,
wherein the particles comprise a single microcrystalline phase; and
wherein at least 5% of the particles are below 3 microns in size.

17. (canceled)
